(19) United States
(12) Patent Application Publication
(10) Pub. No.: US 2008/0052790 A1
(43) Pub. Date: Feb. 28, 2008

(54) COMPOSITION AND METHOD FOR ENHANCING PLANT TRANSFORMATION AND HOMOLOGOUS RECOMBINATION

(75) Inventors: Igor KOVALCHUK, Lethbridge (CA); Alex BOYKO, Lethbridge (CA)

Correspondence Address: SHERIDAN ROSS PC, 1560 BROADWAY, SUITE 1200, DENVER, CO, 80202, US

(73) Assignee: THE UNIVERSITY OF LETHBRIDGE, Lethbridge (CA)

(21) Appl. No.: 11/466,184

(22) Filed: Aug. 22, 2006

(51) Int. Cl. A01H 5/00

(52) U.S. Cl. 800/278; 800/294; 800/317.3; 800/320.3

(57) ABSTRACT

A composition, and method for enhancing transformation of plants is provided. The method involves the use of a transformation-enhancing composition enriched with an effective amount of ammonium nitrate such that, when contacted with plants, it produces an increase in homologous recombination and transformation frequency in the plants. The composition, method and use of ammonium nitrate as described herein reduces undesirable deletions of the introduced DNA.

### FIELD OF THE INVENTION

The present invention relates to plant media and, in particular, to a transformation-enhancing composition for plant transformation and to methods for enhancing transformation frequency and homologous recombination in plants.

### BACKGROUND

Agriculture is a multibillion-dollar industry that can be significantly impacted by even seemingly small improvements in methods or compositions for improving transfer of foreign genes into plants. Traditionally, methodologies based on sexual reproduction have been utilized for the transfer of genes within plant species or between closely related plant species to improve crop qualities. The pace of crop improvement by such methodologies has been slow and limiting, in part due to reliance on naturally occurring genes.

Advances in genetic engineering provide an alternative approach for introducing foreign genetic information into plants, thereby resulting in transgenic plants that have acquired new beneficial characteristics. Genetic engineering of plants involves genetic transformation by introducing foreign genetic material(s) in the form of a nucleic acid such as DNA, which encodes for one or more genes. Other transformation techniques, which are all well known in this field, include somatic hybridization by fusion of protoplasts and the induction of somaclonal variations in order to induce genetic modifications.

The transfer of foreign genetic material into plants is commonly performed utilizing well-known gene transfer techniques such as Agrobacterium-mediated transformation. This technique utilizes strains of Agrobacterium containing an engineered Ti plasmid to introduce the genetic material of interest. These bacteria enable expression of the genetic material and produce transformants or transformed plants that exhibit profitable agronomic characteristics. Thus, it is possible to produce plants with certain desirable characteristics such as resistance to herbicides, insects, and viral diseases.

Large economic expenses have been devoted to the development of recombinant DNA technology for manipulating genetic information in plants. For example, plant genes can be cloned, and desirable genes can be recombined from unrelated organisms to confer new agriculturally useful traits to crops. Recombinant DNA technology has created a larger gene pool available for crop improvement.

However, the benefit of these advances in bioengineering can only be realized if these genes of interest can be introduced into plants reliably, consistently and economically. The enhancement of transformation rate efficiency, even by as much as two-fold, can translate into significant cost savings with respect to expenditures such as technical staff salaries, material costs and energy costs.

There are a number of methods directed to improving plant transformation efficiencies. These methods are aimed at improving the health of the bacteria that is used for transformation, the health of plants that are recipients of transformations and the conditions during plant regeneration.

Plant transformation is by no means a routine matter. For many commercially important crop plants, the efficiency or frequency of transformation as determined by the number of transformed plants produced per each transformation attempt is very low and highly variable from one genetic line or variety to the next. Some highly desirable breeding lines exhibit extremely low transformation frequencies relative to other genetic lines of the same crop species. In some cases, satisfactory levels of transformed plant cells and calli can be achieved from a transformation attempt, but such transformed cells and calli are resistant to regeneration into transformed embryos and plants.

The prior art methods generally result in poor control over where and how the DNA of interest is integrated into the plant genomic DNA during transformation. The introduced genetic material typically integrates randomly and is mediated primarily via non-homologous end joining (described further below), thus leading to frequent inactivity of the transgene and/or modification of the genomic sequences due to integration of truncated copies of the DNA and deletions at the site of integration. Also, the prior art methods are only aimed at improving one of the transformation steps in gene transfer.

It is known that double strand breaks are associated with transformation so that the foreign DNA of interest can integrate into the plant genomic DNA. Repair of the DNA strand breaks are mediated by two major mechanisms or pathways, namely non-homologous end joining (“NHEJ”) and homologous recombination (“HR”). Researchers have revealed that non-homologous end joining is an error-prone mechanism and frequently results in deletions and/or insertions at the place of the repair where the integration has occurred. In contrast, homologous recombination is considered error-free and, therefore, a more desirable mechanism for DNA integration during plant transformation. However, non-homologous end joining is a predominant repair mechanism in plants. It has been shown that the ratio of non-homologous end joining to homologous recombination is at least 1000:1 in plants.

The inability to control where and how genes are integrated and the errors that can be introduced during transformation are major drawbacks of existing methodologies in gene transformation. It is currently unclear what factors control the preferential utilization of non-homologous end joining over homologous recombination for the repair of double strand breaks in plants. Both of these mechanisms play a role in the integration of foreign DNA with respect to transformation. Given that non-homologous end joining is the predominant mechanism utilized in plants, an increase in homologous recombination can lead to more effective integration of the desired gene, more intact “clean” integration and greater control in targeting genes to their desired locations.

It would be advantageous to provide a method and composition that can produce:
an increase in transformation frequency regardless of the method of DNA delivery or transformation methodology employed;
a greater likelihood of transforming “transformation-resistant” plants;
an increase in primary pools of single copy/clean insertions;
a decrease in time and expense required for generation of “clean” and stable transgenic plant lines; and
a reduction in inactivity of the transgene and/or modification of plant genomic sequences, thereby yielding greater efficiency in targeting the introduced DNA to their desired location.

### SUMMARY

In one aspect, a method for transforming plants is provided. The method comprises contacting the plants with a transformation-enhancing composition that contains a plant growth nutrient for growing the plant. The plant growth nutrient is enriched with an effective amount of at least one nitrate-containing compound. A cell from the plant is transformed using the transformation-enhancing composition with a nucleic acid or DNA of interest. A transformed plant is regenerated from the transformed cell.

In another aspect, a method for enhancing plant transformation frequency is provided. The method comprises: a) contacting the plant with an effective amount of a transformation-enhancing composition comprising a plant growth nutrient containing an enriched amount of ammonium nitrate at an effective concentration substantially greater than 1.65 g/L, b) transforming a cell from the plant with a nucleic acid of interest, and c) regenerating a transformed plant from the transformed cell.

In another aspect, there is provided a transformation-enhancing composition for plants comprising a plant growth nutrient for growing the plants, and an effective amount of at least one nitrogen-containing compound, wherein the combination of the nitrogen-containing compound with the plant growth nutrient produces a mixture for enhancing plant transformation frequency.

In another aspect, there is provided a kit for transforming plants, comprising a part A containing a plant growth nutrient for growing plants, and a part B containing an effective amount of an ammonium nitrate for enhancing plant transformation.

In still another aspect, there is provided a use of ammonium nitrate for enhancing plant transformation.

### BRIEF DESCRIPTION OF THE DRAWINGS

The present invention will now be described by way of examples and with reference to the following figures wherein:

FIG. 1A contains panels A and B exemplifying structural arrangements of reporter genes encoding for screenable, or scorable markers beta.-glucuronidase (“GUS”) and luciferase (“LUC”) in DNA constructs used in transformations;

FIG. 1B contains panels C and D depicting representative examples of imaging results obtained for successful homologous recombination after transformation in plants for respectively the GUS and LUC scorable markers;

FIG. 2 illustrates a representative example of a DNA construct structure of the T-DNA in the binary plasmid used in transformations;

FIG. 3 illustrates diagrammatically representative steps in transforming plants and assessing transformation efficiency;

FIG. 4 are photo illustrations depicting the results of transient transformation of tobacco plants germinated and grown for 14 days in the presence of different concentrations of ammonium nitrate. The intensity of blue staining reflects the efficiency of transient transformation;

FIG. 5 is a bar graph depicting the number of calli regenerated per single plant that has been grown in the presence of the transformation-enhancing composition enriched with different amounts of ammonium nitrate;

FIG. 6 is a photo illustration depicting a representative DNA gel with PCR products confirming the transgene integration in plants;

FIG. 7 is a bar graph depicting the percentage of plant transformants that have stably integrated the introduced DNA of interest after being transformed in the presence of the transformation-enhancing composition enriched with different amounts of ammonium nitrate;

FIG. 8 is a photo illustration depicting a representative Southern blot showing integration of “intact” DNA of interest in the plant transformants;

FIG. 9 is a bar graph depicting the percentage of “intact” copies of DNA sequences stably integrated in the plant transformants after being transformed in the presence of the transformation-enhancing composition enriched with different amounts of ammonium nitrate;

FIG. 10 is a bar graph depicting the number of primary embryoides of Triticale regenerated (stable transformation events) per single scuttelum grown with the presence of different concentrations of the amount of ammonium nitrate;

FIG. 11 is a bar graph depicting the number of plant cells expressing anthocyanin pigmentation relative to the amount of ammonium nitrate enriched in the transformation-enhancing composition; and

FIG. 12 is a photo illustration depicting representative tobacco plant leaves with individual stable transformation events (plants regenerated from single transformed cells).

### DETAILED DESCRIPTION OF EMBODIMENTS

In order to promote an understanding and appreciation of the present invention, certain preferred embodiments thereof will be described. It will be understood that only the preferred embodiments are described and that all modifications and further utilizations of the principles of these embodiments as would occur to those ordinarily skilled in the art to which the invention relates are contemplated as being a part of the invention.

Methods, compositions, and kits for enhancing plant transformation are provided. The inventors have demonstrated through experimentation a novel and unexpected finding that enrichment of compounds containing nitrogen such as ammonium-containing compounds, and nitrate-containing compounds in an effective amount to a plant growth nutrient produces a transformation-enhancing composition with desirable qualities for enhancing genetic transformation of plants.

A method for transforming plants is provided. The method generally involves the following steps. Providing a transformation-enhancing composition containing a plant growth nutrient. Enriching the plant growth nutrient with an effective amount of at least one nitrogen-containing compound. Contacting the plant with the transformation-enhancing composition. Transforming a cell from the plant with a nucleic acid or DNA of interest and regenerating a transformed plant from the transformed cell.

In one embodiment, the contacting step involves incubating (described below) an effective amount of the transformation-enhancing composition with the plant, including but not limited to, the plant tissue or cell prior to, during, or after a transformation attempt. The transformation-enhancing composition increases transformation frequency or efficiency when cells from the plant are used in transformation. The method can further comprise transforming the cell from the plant with a nucleic acid or DNA of interest and regenerating a transformed plant.

There are several technologies that can be used to transform or introduce the DNA of interest into the plant. These technologies are well known to those of skilled in the art and can include, but not limited to: (1) chemical methods; (2) physical methods such as microinjection, electroporation, and particle bombardment; (3) viral vectors; (4) receptor-mediated mechanisms; and (5) Agrobacterium-mediated plant transformation methods.

In one embodiment, Agrobacterium-mediated transformation can be used for transforming crop plants. There are several Agrobacterium species that are known, which can mediate the transfer of the DNA, known as “T-DNA”. The T-DNA can be genetically engineered to carry a desired piece of DNA into the selected plant types or species. Some major events marking successful transformation can include, but are not limited to, induction of virulence genes, processing and transfer of the T-DNA to the plant's genome.

Typically, Agrobacterium-mediated genetic transformation of plants involves several steps. The first step involves contacting the Agrobacterium with the plant cells, a process generally known in the art as “inoculation.” In certain occasions known to the skilled artisan, the plant cells are “precultured” before inoculation. This is often to condition the cells to be more amenable to transformation. Following the inoculation step, the Agrobacterium and plant cells/tissues are usually grown together for a period of time suitable for facilitating growth and T-DNA transfer. This step is termed “co-culture” and can be for several hours to several days. Following co-culture and T-DNA delivery, the plant cells are often treated with bactericidal or bacteriostatic agents to prevent further growth of the Agrobacterium. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step.

When a “delay” is used, one or more “selection” steps may follow it. Both the “delay” and “selection” steps typically include bactericidal or bacteriostatic agents to prevent further growth of any remaining Agrobacterium cells because the growth of Agrobacterium cells is undesirable after the infection (inoculation and co-culture) process. Then the selected transgenic cells are put through a “regeneration” step in which transformed plantlets are produced.

It has been contemplated and known to those skilled in the art that there are other methods besides Agrobacterium-mediated transformation for delivery of the DNA of interest into the plant cells. For example different methods can be used to accelerate DNA coated metal particles into living cells. These can include, but not limited to, pneumatic devices; instruments utilizing a mechanical impulse or macroprojectile; centripetal, magnetic or electrostatic forces; spray or vaccination guns; and apparatus based on acceleration by shock wave, such as electric discharge.

In one embodiment, particle bombardment can be used to transform the plant. In this method, particles are coated with the nucleic acid of interest in the form of DNA or RNA. Alternatively, the metal particles may be used to carry the nucleic acid into a cell from a solution of DNA or RNA surrounding the cell. The nucleic acid coated particles are delivered into the plant cells by a propelling force. Exemplary particles include but not limited to those comprised of tungsten, platinum, or, gold.

There are some variables or parameters that can be tested, which are known to those skilled in the art. These include physical parameters such as: (1) the nature, chemical, and physical properties of the metal particles; (2) the nature, preparation, and binding of the DNA onto the particles; and (3) the characteristics of the target plant tissue.

Some environmental variables that have been contemplated include such parameters as temperature, photoperiod and humidity of donor plants, explants, and bombarded tissues.

Some biological factors that have been considered include choice and nature of explant, pre- and post-bombardment culture conditions, and interactions between the introduced DNA and cytoplasmic or nuclear components.

In an illustrative embodiment, a particle delivery system such as the Helios Gene Gun, as manufactured by Bio-Rad Laboratories of Hercules, Calif., U.S.A., can be used to propel particles coated with DNA or cells onto a surface covered with the plant cells cultured in suspension. After bombardment of the plant tissues or cells, selection and regeneration are performed as described above for Agrobacterium transformation.

Typically, to initiate transformation, one selects the nucleic acids or genetic components that is desired to be introduced into the plant cells or tissues. The genetic components can include any nucleic acid that is capable of being introduced into the plant cell or tissue using the method described herein. The genetic components can include non-plant DNA, plant DNA, or synthetic DNA.

In an exemplary embodiment, the genetic components are incorporated into a DNA composition such as a recombinant, double-stranded DNA construct in the form of a plasmid or vector molecule comprising at least one or more of the following types of genetic components: (a) a promoter that functions in plant cells to cause the production of an RNA sequence, (b) a structural DNA sequence and/or functional DNA sequence of interest (described further below) that causes the production of an RNA sequence that encodes a desired protein or polypeptide, and (c) a 3′ non-translated DNA sequence that functions in plant cells to cause the polyadenylation of the 3′ end of the RNA sequence. The DNA construct may also contain a number of genetic components to facilitate transformation of the plant cell or tissue and regulate expression of the desired gene(s).

The genetic components are typically oriented so as to express an mRNA, which can be translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that polyadenylates the 3′ ends of the mRNA.

Means for preparing DNA constructs in the form of plasmids or vectors containing the desired genetic components are well known in the art. DNA constructs in the form of plasmids or vectors typically consist of a number of genetic components, including but not limited to regulatory elements such as promoters, leaders, introns, and terminator sequences. Regulatory elements are also referred to as cis- or trans-regulatory elements, depending on the proximity of the element to the DNA sequences or gene(s) they control.

A region of DNA usually referred to as the “promoter” regulates transcription of the DNA into mRNA. The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells are known in the art. These promoters include, but are not limited to, 35S, 1′/2′, actin, tubulin, and chalcone synthase promoters. All of these promoters can be used to create various types of DNA constructs for expression in plants. Promoter hybrids can also be constructed to enhance transcriptional activity or to combine desired transcriptional activity, inducibility, and tissue or developmental specificity.

Useful promoters may be obtained from a variety of sources such as plants and plant DNA viruses. The particular promoter selected should be capable of causing sufficient expression to result in the production of an effective amount of the gene or DNA product of interest.

The promoters used in the DNA constructs described herein may be modified, if desired, to affect their control characteristics. Promoters can be derived by means of ligation with operator regions, random or controlled mutagenesis, etc. Furthermore, the promoters may be altered to contain one or more “enhancer sequences” to assist in elevating gene expression.

The mRNA produced by a DNA construct may also contain a 5′ non-translated leader sequence. This sequence can be derived from the promoter selected to express the gene and can be specifically modified so as to increase translation of the mRNA. The 5′ non-translated regions can also be obtained from viral RNAs, from suitable eukaryotic genes, or from a synthetic gene sequence. Such “enhancer” sequences may be desirable to increase or alter the translational efficiency of the resultant mRNA.

The described methods and compositions are not limited to DNA construct design or construction. Other genetic components that serve to enhance expression or affect transcription or translation of a gene or DNA sequence are also envisioned as genetic components.

As described above with respect to transformation, the transformed DNA construct or plasmid can contain a selectable, screenable, or scorable marker gene. These genetic components are also referred to as functional DNA sequences, as they produce a product that serves a function in the identification of a transformed plant, or a product of desired utility. The DNA that serves as a selection device functions in a regenerable plant tissue to produce a compound that confers upon the plant tissue resistance to an otherwise toxic compound.

Genes or DNA of interest for use as a selectable, screenable, or scorable marker can include, but are not limited to, beta.-glucuronidase (GUS), green fluorescent protein (GFP), luciferase (LUC), antibiotics like kanamycin, and herbicides like glyphosate. Other selection devices can also be implemented, including, but not limited to, tolerance to phosphinothricin, bialaphos, and positive selection mechanisms.

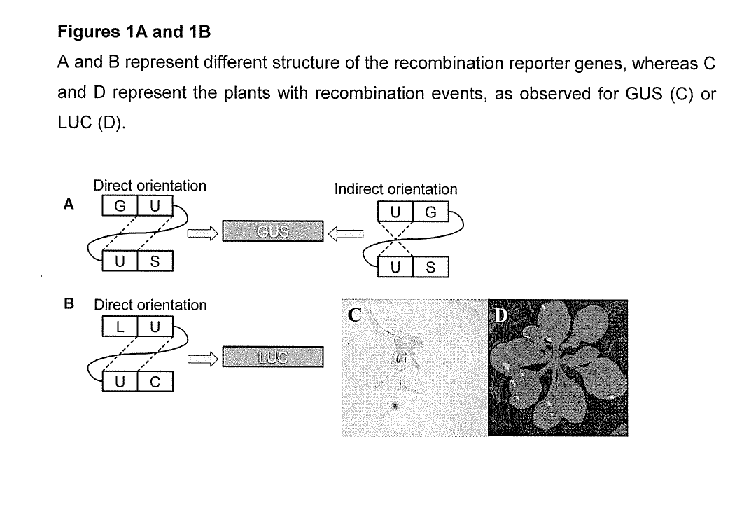

A DNA construct comprising the GUS and LUC reporter markers used to detect homologous recombination is shown in FIG. 1A.

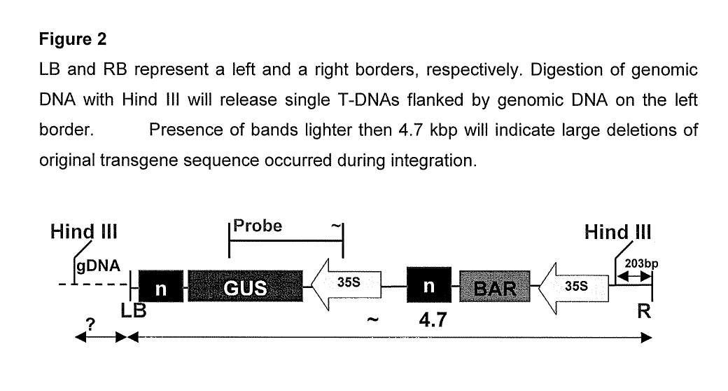

Another DNA construct that is used to assess deletions in the original DNA of interest contained in the DNA construct is shown in FIG. 2.

As disclosed herein, any suitable plant transformation plasmid or vector can be used. The plasmid construct can contain a selectable or screenable marker and associated regulatory elements as described above, along with one or more nucleic acids (a structural gene or DNA of interest) expressed in a manner sufficient to confer a particular desirable trait. Examples of suitable structural genes of interest envisioned would include, but are not limited to, genes for insect or pest tolerance, genes for herbicide tolerance, genes for quality improvements such as yield, nutritional enhancements, environmental or stress tolerances, or genes for any desirable changes in plant physiology, growth, development, morphology, or plant product(s).

Thus, any nucleic acid sequence that produces a protein or mRNA that expresses a phenotype or morphology change of interest is useful for the practice of the present invention. Exemplary nucleic acids that may be introduced by the transformation methods described herein including, for example, DNA sequences or genes from another species, or even genes or sequences that originate with or are present in the same species but are incorporated into recipient cells by genetic engineering methods rather than classical reproduction or breeding techniques.

In one embodiment, a transformation-enhancing composition for plants is provided. The composition contains a plant growth nutrient for growing plants and an effective amount of at least one nitrogen-containing compound. The combination of the compound with the plant growth nutrient produces a mixture for enhancing plant transformation frequency.

The nitrogen-containing compound can include, but is not limited to, nitrate compounds, ammonium compounds, ammonia, ammonium ions, nitrate ions and salts of ammonium and nitrate.

For example, ammonium compounds can include ammonium acetate, ammonium bicarbonate, ammonium bromide, ammonium carbonate, ammonium cerium(IV) nitrate, ammonium chlorate, ammonium chloride, ammonium diuranate, ammonium fluoride, ammonium hexachloroplatinate, ammonium hydrogen fluoride, ammonium hydroxide, ammonium iodide, ammonium lauryl sulfate, ammonium nitrate, ammonium nitrite, ammonium perchlorate, ammonium persulfate, ammonium phosphate, ammonium sulfate, ammonium sulfide, ammonium thioglycolate, ammonium uranyl carbonate, ammonium vanadate, diammonium phosphate.

Nitrate compounds can include, but are not limited to, magnesium nitrate, sodium nitrate, ammonium nitrate, calcium nitrate, barium nitrate, potassium nitrate, hydroxylammonium nitrate, bergilium nitrate, caesium nitrate, chromium nitrate, cobalt nitrate, ferric nitrate, lead nitrate, lithium nitrate, manganese nitrate, mercury nitrate, nickel nitrate, silver nitrate, strontium nitrate, and zinc nitrate.

The transformation enhancing composition as used herein, refers to the plant tissue culture media, whether liquid, solid, or semisolid, used before, during, or after the transformation of the plant cells, tissues, parts, or other plant tissue explants and subsequent regeneration of whole, transgenic plants therefrom. Depending upon the plant species being transformed and the transformation process being used, the transformation media may include, but is not limited to, the isolation media, preculture media, induction media, inoculation media, delay media, selection media, or regeneration media.

The plant cells or tissues can include, but are not limited to, immature embryos, scutellar tissue, suspension cell cultures, immature inflorescence, shoot meristem, nodal explants, callus tissue, hypocotyl tissue, cotyledons, roots, and leaves.

Desirable results in transformation are achieved with an enrichment of the nitrogen-containing compound in the form of ammonium nitrate to the plant growth nutrient wherein the concentration of the ammonium nitrate is greater than 1.65 g/L.

In one embodiment, the enrichment of the plant growth nutrient with ammonium nitrate produces a transformation-enhancing composition that substantially enhances plant transformation, particularly transformation frequency and/or homologous recombination.

In another embodiment, the amount of ammonium nitrate is greater than 1.65 g/L and less than 5.00 g/L.

In another embodiment, the amount of ammonium nitrate is greater than 1.65 g/L and less than or equal to 3.30 g/L.

It is observed that replacement of the ammonium ion or the nitrate ion decreases the enhancement of transformation, particularly transformation frequency and/or homologous recombination.

In one embodiment, the plants are grown in the transformation-enhancing composition in the presence of the Agrobacterium in the form of an Agrobacterium suspension for fourteen days under sterile conditions. The Agrobacterium contains a screenable, or scorable marker gene encoding for GUS for measuring transformation and a selectable marker, BAR, for selection of transformants resistant to a herbicide BASTA.

In one embodiment, Arabidopsis plants are grown on the transformation-enhancing composition. It has been shown that this produces more than five-fold enhancement of transient transformants. The transformation-enhancing composition also resulted in a substantial increase in transient transformation of Tobacco plants by about four- to about twenty-fold. The number of transformed calli in the Tobacco plants have been shown to increase two- to three-fold, and the number of regenerated transgenic plants increased from about 60% to about 80%. Examination of integration events in transgenic plants showed a statistically significant increase in the acquisition of intact integrated T-DNA from Agrobacterium into the plants relative to control plants (described below).

Typically, an effective amount of the transformation-enhancing composition may be determined by contacting an amount of the transformation-enhancing composition with a first plant and transforming cells from the plant with a DNA construct. For a comparison, cells from a second plant, referred to as the control plant, to which the transformation-enhancing composition was not administered, are transformed with the same DNA construct at approximately the same time. Typically the results of such transformation attempts are indicated as the number of transformed cells, calli, embryos or plants. A comparison of the results achieved with cells from the first and second plants is used to determine whether the amount of the transformation-enhancing agent administered is an effective amount. By this approach, an effective amount of a transformation-enhancing agent is an amount sufficient to, provide an increased number of transformed cells, calli, embryos or plants when administered to the plant, relative to the number of transformed cells, calli, embryos or plants achieved with the control plant.

In one embodiment, wheat plants are transformed using particle bombardment as described above using gold particles to carry the DNA of interest. Gold-particle bombardment is performed in the presence of the transformation-enhancing composition. The gold particle carries the DNA encoding for antocyanin production to embryonic tissues of the wheat plants.

It has been shown that a 2-day incubation of the wheat embryonic tissues, such as its scuttelum, in the presence of the transformation-enhancing composition containing 5.00 g/L of ammonium nitrate resulted in a 25% increase in transformation frequency or efficiency relative to control plants.

In one embodiment, Arabidopsis or tobacco plants are grown on the transformation-enhancing composition.

The compositions and methods provided herein find use in transforming plant cells and regenerating transformed or transgenic plants. The compositions and methods provided herein are not specific for particular plant types or species, but can be used for enhancing transformation in a variety of plants, including but not limited to, monocot and dicot plants, and in particular to Arabidopsis, wheat, cereal, and tobacco plants.

The transformation-enhancing composition, when provided to the plant in an effective amount prior to, during, or after a transformation attempt, increases the frequency or efficiency of the transformation.

While the transformation-enhancing composition does not depend on any particular biological mechanism for increasing the transformation efficiency, it is recognized that the transformation-enhancing composition may alter one or more processes such as, for example, uptake of foreign DNA or nucleic acid by a cell, integration of foreign DNA or nucleic acid into the genome of a cell, expression of a foreign gene or DNA in a transformed cell, proliferation of a transformed cell, cell division, callus formation, embryogenesis, root formation, shoot formation and leaf formation. The transformation-enhancing composition can affect any one or more of the structures, mechanisms or processes of the plant in such a manner that transformation efficiency or frequency is enhanced.

In addition, the transformation-enhancing composition may find use in initiation, growth and differentiation of cultures of plant cells and plant tissues. These plant cultures can comprise of plant cells that are genetically transformed, untransformed or both. The transformation-enhancing composition can enhance one or more desired processes associated with cultures of plant cells and plant tissues including, but not limited to, cell culture initiation, cell proliferation, callus formation, embryogenesis, differentiation, shoot formation, leaf formation and root formation.

As described earlier, the methods and compositions provided herein find use in improving transformation frequency or efficiency, even with plants known to be resistant to transformation and regeneration by existing methods.

In one embodiment there is provided a method comprising contacting the transformation-enhancing composition with the plant prior to or during a transformation attempt with the DNA construct or nucleic acid of interest.

The DNA construct or nucleic acid of interest can be delivered to the plant cell as described above, or by any means for transforming the plant known to those skilled in the art, including, but not limited to, Agrobacterium-mediated, microprojectile bombardment, direct protoplast transformation, infiltration, electroporation of cells and tissues of plants, electrophoresis of embryos, microinjection, pollen-tube pathway, silicon carbide- and liposome-mediated transformation.

The transformation-enhancing composition can be contacted with the plant as described above, or by any means known to those skilled in the art. This can include, for example, direct incubation, growth in the composition, external application of the composition to the plant body in sprays, drips, mists, powders, dusts, vapors or gases, injecting the plant or any part thereof with the composition, soil application and application in irrigation water.

The transformation-enhancing composition may be contacted with the plant for any length of time suitable to produce enhanced transformation efficiency. The time of plant contact with the composition can vary, depending on the plant type, plant species, or plant tissue that is utilized for transformation.

In one embodiment, tobacco and Arabidopsis plants were germinated and grown on the composition for approximately two weeks, whereas in another embodiment, tobacco plant leaves were incubated on the composition for 3 days.

In another embodiment, triticale scuttela were exposed to the composition for 36 hours. It has been shown that contacting the plant with the composition for 36 hours is sufficient to produce desirable results, although longer contact times is preferable.

The transformation-enhancing composition can be contacted with a specific part of the plant body. The specific part of the plant body may contain the desired cells for transformation with the DNA construct or nucleic acid of interest.

In one embodiment, the transformation-enhancing composition can be contacted with a part of the plant body, which does not contain the desired cells for transformation to achieve an increase in transformation efficiency. The increase in transformation efficiency may result from active or passive transport or diffusion of the transformation enhancing composition to the desired cells for transformation.

In another embodiment, the transformation-enhancing composition may affect cells in the region where the transformation-enhancing composition was administered resulting in the affected cells initiating or facilitating in some manner one or more structural or physiological changes in the plant that lead to an increase in transformation efficiency or frequency in the desired cells for transformation.

It is well described in the art that homologous recombination is involved in efficient integration the introduced nucleic acid or DNA of interest to their desired location in the plant genome. It is also known to those skilled in the art that homologous recombination is much more effective than non-homologous end joining for increasing the chance for the nucleic acid or DNA to be inserted in the desired location, in an unaltered and undeleted form. Stated another way, homologous recombination is more desirable with respect to plant transformation for increasing the likelihood of effective gene targeting.

It has been shown that the transformation-enhancing composition disclosed herein increases the frequency of homologous recombination without increasing the level of double-strand breaks. It has also been shown that the transformation-enhancing composition resulted in an enhancement of homologous recombination and substantially increased the proportion of homologous recombination relative to non-homologous end joining.

Accordingly, it is predicable and envisioned that the transformation-enhancing composition as disclosed herein can increase effective targeting of the introduced DNA to their desired location, due to a reduction in the incident of non-homologous end joining.

### EXAMPLES

The following examples further illustrate the methods and composition described herein.

### Example 1

Preparation of DNA Constructs and Analysis of Nitrogen-containing Compounds on Homologous Recombination.

DNA constructs were prepared according to standard molecular biology techniques known to those skilled in the art. FIG. 1A shows panels A and B depicting exemplary structural arrangements of DNA constructs containing the reporter markers GUS and LUC. Recombination between the regions of homology (depicted as “U”) results in the restoration of the introduced transgene (i.e. GUS or LUC) in the transformed plants. This approach allows analysis of homologous recombination events in the plants transformed in the presence or absence of various nitrogen-containing compounds.

Homologous recombination was detected in plants, in particular Arabidopsis and tobacco plants, that have been transformed with the scorable reporter marker beta.-glucuronidase (uidA or GUS). Homologous recombination was scored by standard histochemical staining or other imaging techniques known to those skilled in the art. An exemplary imaging result is shown as brightened blue regions on the transformed plants (see FIG. 1B). This approach allows quantitative assays to be undertaken as described in Boyko et al., (2006), which is incorporated herein in its entirety.

FIG. 2 shows the DNA construct that is used to assess the efficiency of integration, in particular deletions of the original transgene after transforming the plants. The presence of bands having a molecular size less than 4.7 kbp would indicate an undesirable deletion of the original nucleic acid or DNA of interest during integration.

The influence of various transformation-enhancing composition on homologous recombination frequency was analyzed by changing (i.e. eliminating or increasing) the amount of nitrogen-containing compounds or ammonium-containing compounds in the plant growth nutrient. In an exemplary embodiment, the Murashige Skoog (“MS”) medium is used. Other plant growth media known to those skilled in the art can be used, such as the Gamborg's B5 medium or the Chu's N6 medium. It should be noted that these plant media do not contain ammonium nitrate.

Various nitrogen-containing compounds were analyzed for their affect on transformation efficiency, in particular homologous recombination frequency using the DNA constructs described above. It was demonstrated that ammonium nitrate was the factor that exhibited the largest increase in homologous recombination frequency. Ammonium nitrate was further analyzed in terms of the concentration that produced the most efficient transformation.

### Example 2

Analysis of Ammonium Nitrate Concentration and Homologous Recombination

The affect of ammonium nitrate enrichment of MS-medium on homologous recombination frequency was analyzed. In exemplary experiments, ammonium nitrate concentrations were varied, from 0.0001x to 10.0x, where “x” is equivalent to 1.65 g/L concentration in the MS-medium. Although ammonium nitrate in the amount greater than 1.65 g/L, and notably in the range of greater than 1.65 g/L and less than or equal to 5.00 g/L produced desirable enhancement of homologous recombination frequency, the amount of ammonium nitrate producing the greatest increase in homologous recombination frequency with little or no change in plant appearance was in the range greater than 1.65 g/L and less than or equal to 3.30 g/L.

For the transformation experiments, Arabidopsis and tobacco plants were germinated and grown on the MS-medium containing either 1.65 g/L (1×), 3.30 g/L (2×) or 5.00 g/L (3×) of ammonium nitrate. Both tobacco and Arabidopsis plants were incubated with Agrobacterium carrying the plasmid with GUS (gene coding for β-glucuronidase) and BAR (gene coding for the resistance to phosphinothrycin (herbicide BASTA) genes at 14 days after germination.

For transformations, an Agrobacterium strain GV3101 (i.e. AtvirD2), as described in Tinland et al., (1995), carrying a selectable marker encoding for a gene product for conferring resistance to an antibiotic rifampycin was transformed with a pJL513 DNA construct comprising the GUS and BAR genes operably linked to the control of 35S and 1′/2′ promoters, respectively. The GUS and BAR genes are cloned in between two T-DNA borders, allowing the processing by the Agrobacterium and delivery of the entire T-DNA portion.

Transformed Agrobacteria were selected by growing them in a media containing 50 μg/ml of spectinomycin. The Agrobacteria that were resistant to the spectinomycin were selected, re-plated on spectinomycin-containing media and the resulting colonies were used to start a 4-mL liquid culture (containing YEB medium with 10 mM magnesium sulfate, 100 μg/mL of ryphampycin, and 50 μg/mL of spectinomycin).

The liquid culture was incubated overnight and 500 μL of the Agrobacteria culture was used to prepare a 100-mL culture. Agrobacteria cultures having densities in a selected range having optical densities in the range of about 1.5 to about 2.0 were collected, and washed with 10 mM magnesium sulfate. A pellet obtained after washing steps was resuspended in 50 mL of a growth medium having a pH of about 5.2. This Agrobacterium suspension was used for vacuum infiltration.

Approximately 300 Arabidopsis or tobacco plants grown on 1×, 2×, or 3× ammonium nitrate enriched transformation-enhancing compositions were vacuum infiltrated according to standard procedures known in the art for 2 times for 5 min with the Agrobacterium suspension culture.

After vacuum infiltration, the plants were dried on sterile filter paper to remove substantially the Agrobacteria. The plants were placed at 22° C. and exposed to light for approximately 16 hours, followed by 8 hours in the absence of light. This cycle was carried out for 3 days on the growth media.

Transformations were examined approximately 3 days after the vacuum infiltration step. Approximately ⅔ of the plants from each experimental group (1×, 2×, or 3×) were washed with sterile water to remove the Agrobacterium suspension. The transformed plants were used for histochemical staining by placing them into 1× X-gluc (5-bromo-4-chloro-3-indolyl-beta-D-glucuronic acid, cyclohexylammonium salt) solution. Cleavage of X-gluc by the GUS results in formation of insoluble blue precipitate.

After vacuum infiltration, the plants can also be incubated in X-gluc for 48 hours at 37° C. To analyze the GUS expression, the chlorophyll was washed away by 100% ethanol and blue patterns on the transformed plants were analyzed by counting the number of cells containing the blue patterns per each of the plants.

To decrease the number of transformed cells, the Agrobacterium strain 31001 containing the GUS gene was diluted with a strain of Agrobacterium (i.e. VirD2-), which is deficient in transformation. Dilutions with the VirD2-Agrobacterium strain were performed in 1:10, 1:100, 1:1000, and 1:10000. In an exemplary experiment, the results showed that a 1:1000 concentration was optimal for transient transformation assays.

### Example 3

Stable Transformations and Integration of the Introduced DNA into the Plant Genome

The next example describes stable transformations where the DNA of interest has stably integrated into the plant genome. At least 100 plants per each experimental group (1×, 2×, and 3×) were incubated for 3 days with undiluted Agrobacteria carrying the GUS gene. They were washed with sterile water containing rifampycin. The washed plants were transferred to CIM media (MS media having a pH of about 5.7, and containing 2,4-D of 1 mg/L and kinetin of 0.2 mg/L). The CIM media further comprising 10 mg/L of an antibiotic phosphinothrycin. Plants were moved to fresh CIM plates every 2 weeks. When calli material developed, typically in 5-6 weeks, plants were transferred to SIM media (MS media containing naphthaleneacetic acid (NAA) of 0.1 mg/L and benzylaminopurine (BAP) of 1 mg/L for shoot induction. The number of green regenerated calli, resistant to phosphinothrycin, was counted approximately 6 weeks after transfer to CIM media. Newly appeared plantlets that started forming roots were transferred to soil.

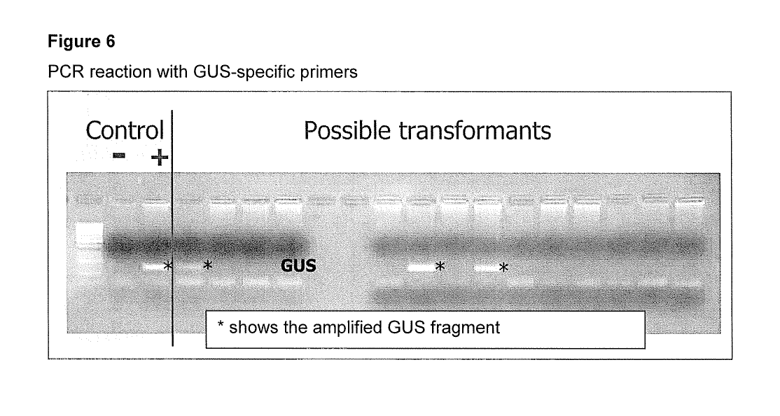

Single leaves of five-week-old plants were used for the PCR analysis. Primers specific for the GUS gene were used for amplification. The number of PCR-positive plants was used for calculation of stable integration frequency as representatively shown in FIG. 6.

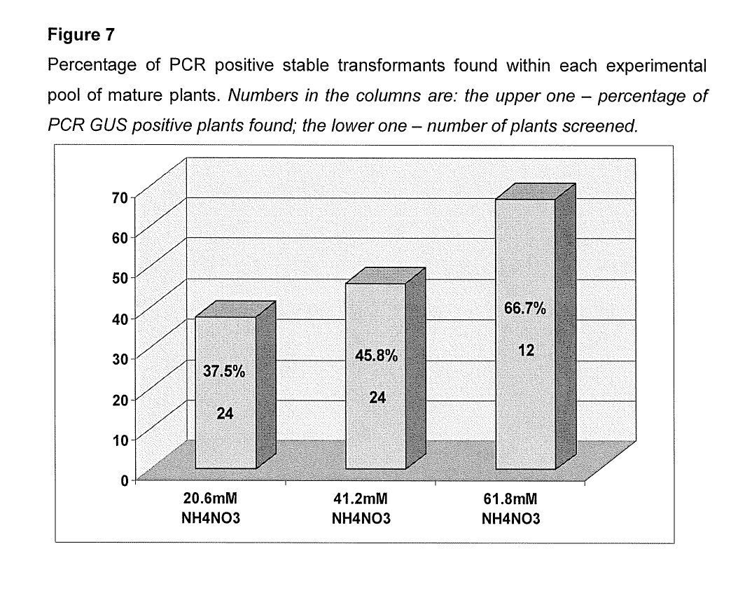

Seeds from plants that scored positive upon PCR (as representatively illustrated in FIG. 7) were collected and used for segregation analyses. Segregation analyses were performed by spraying the 3-day, 6-day and 10-day-old plants with BASTA (phosphinothricin-3-D-glufosinate ammonium in detergent).

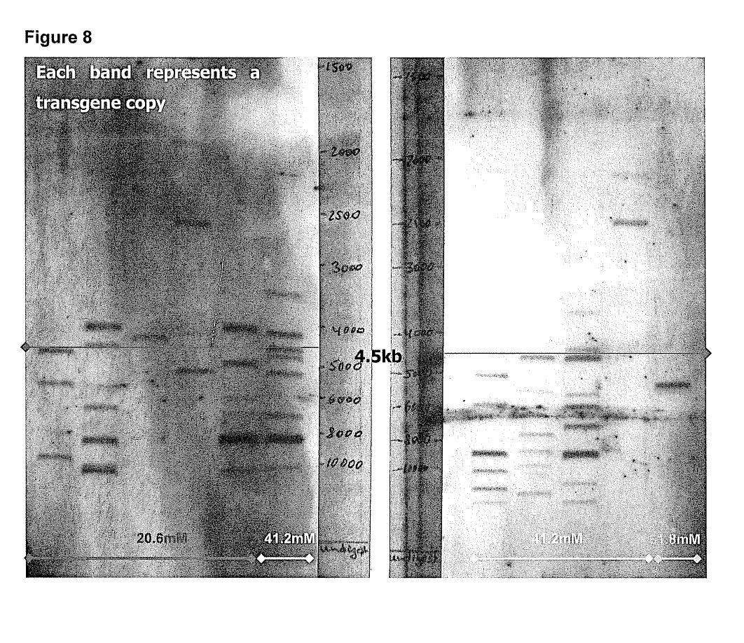

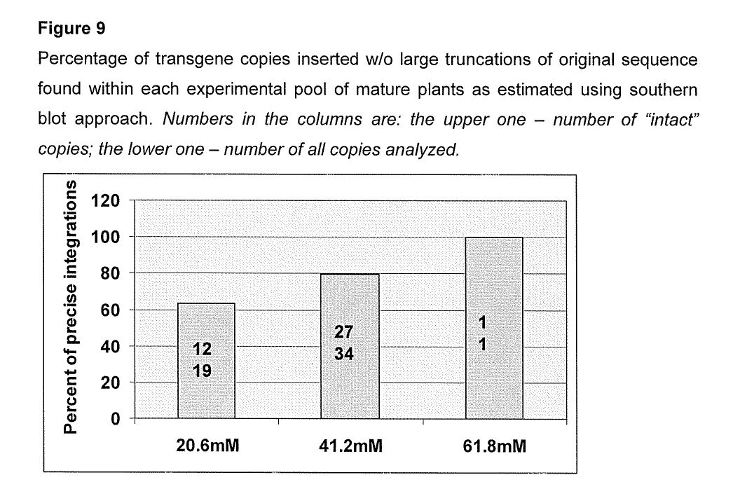

Segregation analyses determined the number of plants with single locus insertion. The DNA from these plants was used for Southern blot analysis and the results are representatively shown in FIG. 8. Southern blots were performed by digestion of genomic DNA with HindIII. This enzyme cuts the T-DNA close to the right border (see FIG. 2). This allows examination of the number of integrated transgenic copies as representatively shown in FIG. 9. The probe consisted of 1 kbp of the promoter and 5′ end of the GUS gene.

Given that the distance from the Hind III recognition site to the left border is 4.5 kb, it follows that the intact integrated DNA that is introduced should be at least 4.5 kb in length (see FIG. 2). DNA introduced in the plants that are less than 4.5 kb in length are considered truncated or deleted, indicating inefficiencies in the transformation. This allows analysis of the percentage of intact DNA integrations into the plant genome. The results of such experiments are representatively shown in FIGS. 9,10 and 11.

### Example 4

Demonstration of Enhanced Transformation Efficiency

Transient transformation of Arabidopsis plants showed a three to four-fold increase in the number of transient transformation events. There were 28 events per 202 plants that were grown on 1× transformation-enhancing composition, 52 events per 178 plants grown on 2× transformation-enhancing composition and 103 events per 197 plants grown on 3× transformation-enhancing composition.

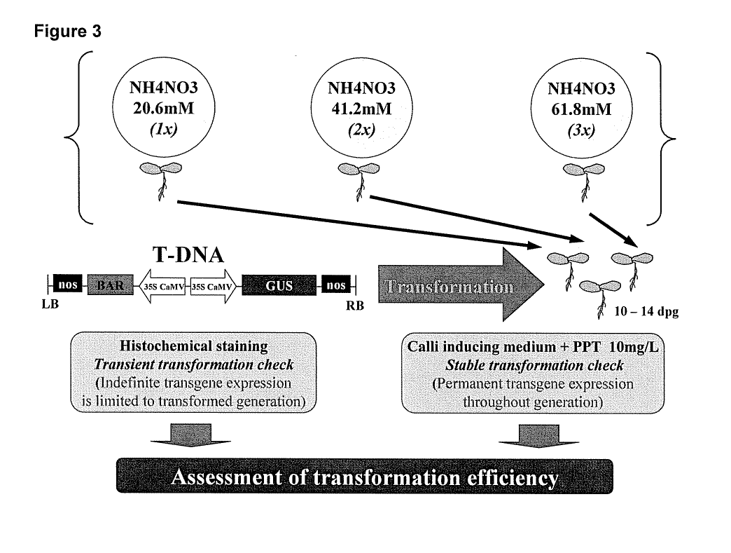

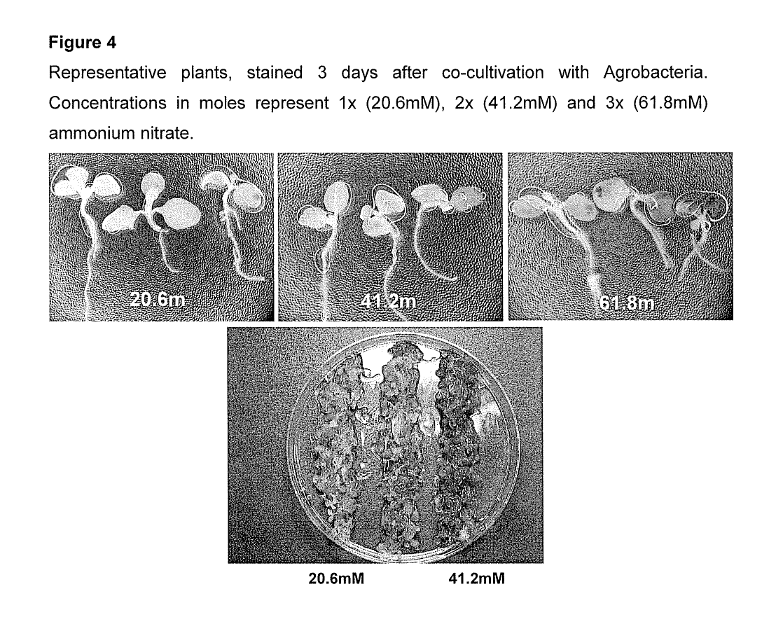

The approach in transforming tobacco plants is shown in FIG. 3. Tobacco plants were transformed with Agrobacterium suspension comprising 1 unit of GUS-containing Agrobacteria and 100 units of VirD2-containing Agrobacteria deficient in transformation. The recombination events were quantitatively measured by scoring bright blue sectors on the plants. The plants that contained a single discrete homologous recombination event were used for counting. The results demonstrated substantial differences in the number of transformation events for plants transformed in 1× transformation-enhancing composition compared to those transformed in 3× ammonium nitrate enriched transformation-enhancing composition, as representatively shown in FIG. 4.

Nearly 50% of all transformed plants grown on 3× ammonium nitrate enriched transformation-enhancing composition were positive for more than 1 transformation event. In contrast, less than 20% of plants grown on 1× transformation-enhancing composition were positive for more than 1 transformation event. The plants that contained a single homologous recombination event were scored. It was shown that plants transformed in the presence of 3× ammonium nitrate enriched transformation-enhancing composition had more than 20 homologous recombination events per plant, whereas the 1× transformation-enhancing composition had 5-7 homologous recombination events per plant. The overall results demonstrated that the plants transformed in the presence of the 3× ammonium nitrate enriched transformation-enhancing composition exhibited more than four-fold difference in homologous recombination events.

Experiments utilizing 1:1000 dilution demonstrated that 3 plants out of 51 plants that were grown on 1× transformation-enhancing composition contained bright blue sectors. In contrast, 7 out of 54, and 11 out 50 plants that were transformed in the presence of respectively 2× and 3× ammonium nitrate enriched transformation-enhancing composition resulted in bright blue sectors. The difference in the number of individual homologous recombination events was also substantial. There were 3.1 events (i.e. 159/51) on the 1× transformation composition and 4.2 (212/50) events on the 3× ammonium nitrate enriched transformation-enhancing composition. It is reasonable to conclude based on these exemplary experiments that transformation frequencies are relatively higher in plants transformed in the presence of the 3× ammonium nitrate enriched transformation-enhancing composition. The enhanced transformation frequencies observed are typically in the range of four- to twenty-fold increases.

### Example 5

Regeneration of Stable Transformants

Plants that have been transiently transformed with 1:100 diluted Agrobacteria strain were used for regeneration of stable transformants. When the numbers of regenerated calli were compared between the 1×, 2×, and 3× transformation media, it was demonstrated that there was a 2.4-fold more calli obtained from the 3× ammonium nitrate enriched transformation-enhancing composition.

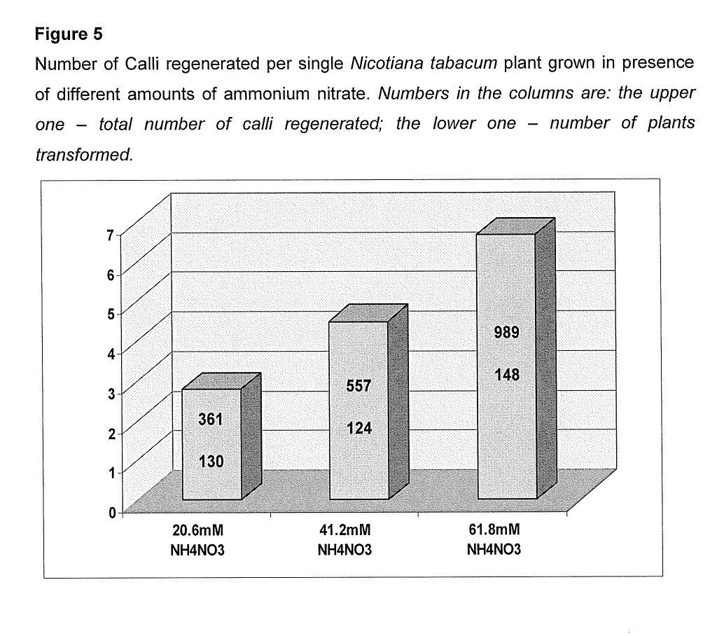

Referring now to FIG. 5, which shows the results of representative experiments. On average per single transformed plant, it is possible to regenerate 2.78 calli for the 1× transformation-enhancing composition, 4.5 calli for the 2× transformation-enhancing composition, and 6.68 calli for the 3× transformation-enhancing composition.

### Example 6

Demonstration of Integration of Intact DNA into Recipient Plants

FIG. 6 shows the results of representative experiments in PCR analysis of plant tissues that have been transformed in the presence of 1×, 2× and 3× ammonium nitrate enriched transformation-enhancing composition. The combined results as shown in FIG. 7 demonstrated a 60% to 70% difference, and about 60% to about 80% more PCR-positive plants transformed in the 3× transformation-enhancing composition, as compared to the 1× transformation-enhancing composition.

Segregation analysis performed by spraying plants with BASTA revealed no significant difference in the number of lines with 3:1 (single locus) segregation.

Analyses by Southern blot allowed determination of the number of “intact” or untruncated copies of the introduced gene or DNA that have stably integrated into the plant genomic DNA. FIG. 8 shows that all the introduced T-DNA copies that are smaller than 4.7 kilobase can be considered truncated or deleted.

The combined results as illustrated in representative experiments of FIG. 9 demonstrated that there were more intact T-DNA copies in transgenic plants transformed in the 2× and 3× ammonium nitrate enriched transformation-enhancing compositions as compared to the 1× transformation-enhancing composition.

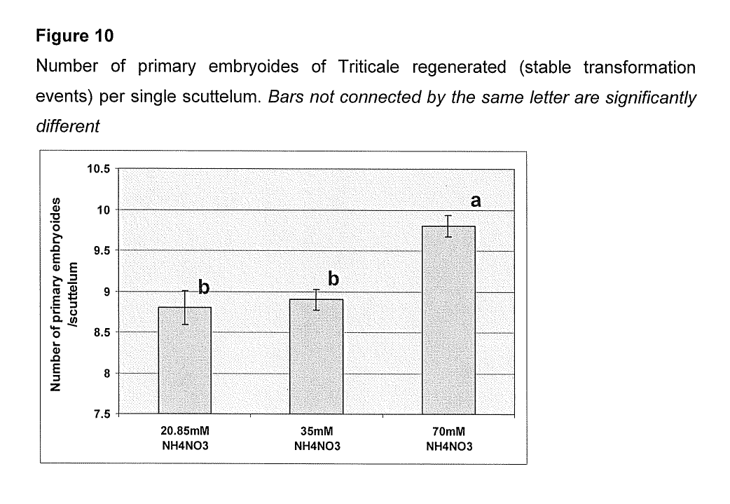

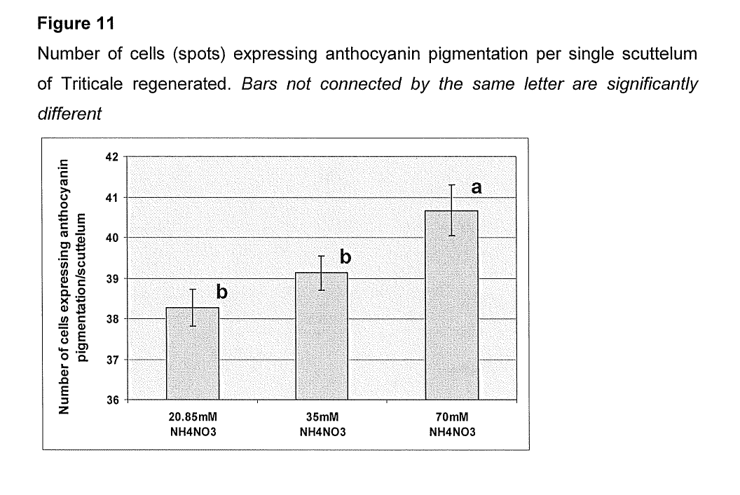

Results from triticale transformation revealed that scuttela that were incubated on the 3× ammonium nitrate enriched transformation-enhancing composition have responded with approximately 10% increase in the number of primary embryoides and cells expressing anthocyanin pigmentation (see FIGS. 10 and 11). This increase has been shown to be statistically significant (as determined from the average data from 300 scuttela per treatment group).

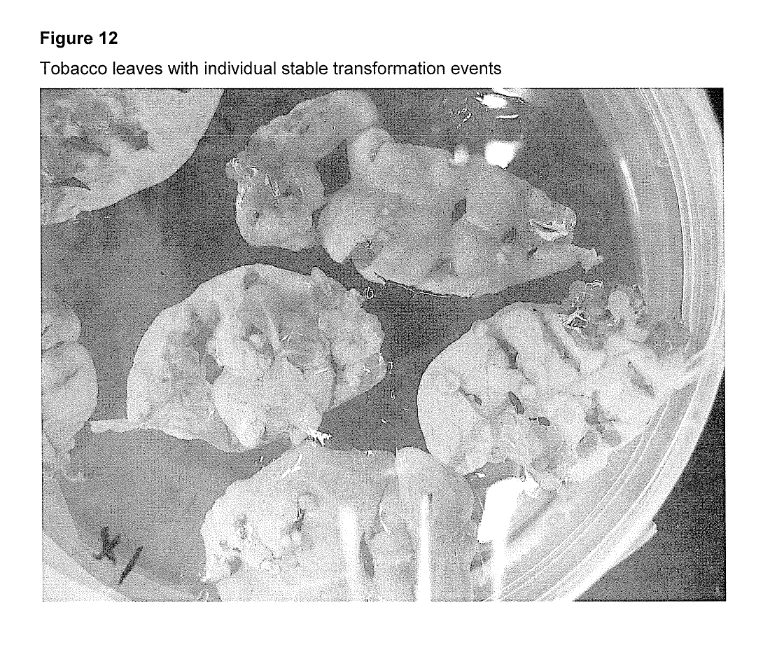

FIG. 12 shows a representative photo illustration of tobacco plant leaves with individual stable transformation events. Each transformed cell gives rise to an individual plant. The number of regenerated plant per single transformed leaf reflects the transformation frequency.

### Example 7

Transformations in Tobacco Plants

Various transformation-enhancing compositions were tested and the results of the experiments are shown below in Table 1 and Table 2. In the experiment of Table 1, tobacco plants were transformed in the presence of 1× and 5× ammonium nitrate enriched transformation-enhancing compositions. The results showed 100 percent LUC expression for both the 1× and 5× ammonium nitrate enriched transformation-enhancing compositions and 60 and 46 LUC positive calli, respectively. PCR analysis revealed integration frequencies of 3.00 and 1.44, respectively for 1× and 5× ammonium nitrate enriched transformation-enhancing compositions.

TABLE 1

| | | Calli regenerated and transplanted | | | | Integration |
|---|---|---|---|---|---|---|
| | | CCD LUC | | | | frequency |
| | | expression test | | | | LUC positive |
| | leaves | LUC | LUC | | died | plants/ |
| | transformed | positive | negative | total | on soil | leaf transformed |
| 1x | 20 | 60 | 0 | 60 | 5 | 3.00 |
| 5x | 32 | 46 | 0 | 46 | 6 | 1.44 |

In the experiment of Table 2, tobacco plants were transformed in the presence of 1×, 3×, and 5× ammonium nitrate enriched transformation-enhancing compositions. Transformations in the presence of 1×, 3× and 5× ammonium nitrate enriched transformation-enhancing compositions produced respectively 5.0, 12.33, and 1.38 LUC positive plants/transformed plant. The combined results indicated that the 3× ammonium nitrate enriched transformation-enhancing composition is more effective than the 5× ammonium nitrate enriched transformation-enhancing composition in Tobacco plants.

TABLE 2

| | | | | Calli regenerated and | | | | Integration |
|---|---|---|---|---|---|---|---|---|
| | | Calli regenerated | | transplanted to soil | | | | frequency |
| | | | Regeneration | CCD LUC expression | | | | LUC positve |
| | | | frequency | test | | | died | plants/ |
| | plants | | on CIM | LUC | LUC | | on | plant |
| | transformed | Number | calli/plant | positive | negative | total | soil | transformed |
| 1x | 7 | 49 | 7.00 | 35 | 0 | 35 | 5 | 5.00 |
| 3x | 9 | 162 | 18.00 | 111 | 0 | 111 | 43 | 12.33 |
| 5x | 8 | 25 | 3.13 | 11 | 0 | 11 | 9 | 1.38 |

Definitions

A number of terms used herein are defined and further clarified below. Where terms that have not been defined herein, they should be given the common meaning that would be understood by those skilled in the relevant art or science to which this invention pertains.

As used herein, the phrase “increase or enhance transformation frequency” refers to an increase of the number of transformed plants recovered from a transformation attempt where the increase is at least 2-fold.

As used herein, the phrase “transformation enhancing composition” refers to a composition that when applied or contacted with a plant before, and/or after, and/or during a transformation attempt increases transformation frequency. Such a “transformation enhancing composition” may favorably influence one of more of the physiological processes associated with genetic transformation and the subsequent regeneration of transformed plants including, but not limited to, foreign DNA uptake into a plant cell, foreign DNA integration in a genome of the plant cell, plant cell proliferation in culture, plant cell culture initiation, proliferation of transformed plant cell, embryogenesis, shoot initiation and root initiation.

As used herein, the term “effective amount” relating to the transformation-enhancing composition refers to an amount that favorably affects one or more of processes associated with increasing transformation frequency including, but not limited to, uptake of foreign DNA by a cell, integration of foreign DNA into the genome of a cell, expression of a foreign gene in a transformed cell, proliferation of a transformed cell, callus formation, embryogenesis, root formation and shoot formation. Those of ordinary skill in the art understand that such an “effective amount or ratio” depends on a number of factors including, but not limited to, the transformation-enhancing composition that is employed, the species of plant, the developmental stage of the plant, the method by which the transformation-enhancing composition is utilized, environmental conditions and the type of plant cells to be transformed.

As used herein, the term “regeneration” or “regenerating” refers to the ability to grow the non-transgenic or transgenic (transformed) plants from a single transformed cell. This term can refer to the totipotency of plants, the ability to regenerate an entire plant from any plant cell. The efficiency depends on a number of factors; thus different cells (organs) of the plant have different regeneration efficiency.

As used herein, the term “enriching” refers to increasing the concentration of a nitrogen-containing compound, such as magnesium nitrate, sodium nitrate, ammonium nitrate, calcium nitrate, barium nitrate, potassium nitrate, hydroxyl ammonium nitrate, bergilium nitrate, caesium nitrate, chromium nitrate, cobalt nitrate, ferric nitrate, lead nitrate, lithium nitrate, manganese nitrate, mercury nitrate, nickel nitrate, silver nitrate, strontium nitrate, and zinc nitrate, in an effective amount greater than 1.65 g/L so as to result in enhanced transformation efficiency and/or homologous recombination.

As used herein, the term “DNA of interest” refers to single- or double-stranded DNA that is used for integration into the plant genome. This can be either the DNA sequence of the gene that is used to improve particular trait or any piece of DNA to be integrated into plant genome.

As used herein, the term “foreign” relating to nucleic acids refers to nucleic acid sequences, DNA or genes that are not normally present in the cell being transformed or to nucleic acid sequences or genes that are not present in the form, structure, etc., as found in the transforming DNA segment or to nucleic acid sequences or genes that are normally present but a different expression is desirable. Thus, the term “foreign” DNA or gene is intended to refer to any DNA or gene segment that is introduced into a recipient plant cell, regardless of whether a similar gene may already be present in the plant cell. The type of DNA included in the foreign DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

It will be noted that the embodiments disclosed herein are well adapted to attain all the ends hereinabove set forth together with other advantages which are obvious and which are inherent to the disclosed process. Many embodiments may be made of the methods and compositions described herein without departing from the scope of the invention. Accordingly, it is to be understood that all matter herein set forth is to be interpreted as illustrative. Certain features and subcombinations that are of utility may be employed including substitutions, modifications, and optimizations, as would be available expedients to those of ordinary skill in the art. The terms and expressions used in the preceding specification have been used herein as terms of description and not of limitation, and there is no intention in the use of such terms and expressions of excluding equivalents of the features shown and described or portions thereof, it being recognized that the scope of the invention is defined and limited only by the claims that follow.

### REFERENCES

1. Boyko, A., Filkowski, J., Kovalchuk, I. (2006) Double strand break repair in plants is developmentally regulated. Plant Physiology, 141, 1-10.
2. Belzile F J. (2002) Transgenic, transplastomic and other genetically modified plants: a Canadian perspective. Biochimie. 84(11):1111-8.
3. Friesner J, Britt A B. (2003) Ku80- and DNA ligase IV-deficient plants are sensitive to ionizing radiation and defective in T-DNA integration. Plant J. 34(4):427-40.
4. Gallego M E, Bleuyard J Y, Daoudal-Cotterell S, Jallut N, White C I. (2003) Ku80 plays a role in non-homologous recombination but is not required for T-DNA integration in Arabidopsis. Plant J. 35(5):557-65.
5. Gelvin S B. (2003) Agrobacterium-mediated plant transformation: the biology behind the “gene-jockeying” tool. Microbiol Mol Biol Rev. 67(1):16-37.
6. Hanin M, Paszkowski J. (2003) Plant genome modification by homologous recombination. Curr Opin Plant Biol. 6(2):157-62.
7. Hanin M, Volrath S, Bogucki A, Briker M, Ward E, Paszkowski J. (2001) Gene targeting in Arabidopsis. Plant J. 28(6):671-7.
8. Kumar S, Fladung M. (2001) Controlling transgene integration in plants.
9. Li J, Vaidya M, White C, Vainstein A, Citovsky V, Tzfira T. (2005) Involvement of KU80 in T-DNA integration in plant cells. Proc Natl Acad Sci USA. 102(52):19231-6.
10. Ninomiya Y, Suzuki K, Ishii C, Inoue H. Highly efficient gene replacements in Neurospora strains deficient for nonhomologous end-joining. Proc Natl Acad Sci USA. 101(33):12248-53.
11. Shaked H, Melamed-Bessudo C, Levy A A. (2005) High-frequency gene targeting in Arabidopsis plants expressing the yeast RAD54 gene. Proc Natl Acad Sci USA. 102(34):12265-9.
12. Tinland B, Schoumacher F, Gloeckler V, Bravo-Angel A M, Hohn B. (1995) The Agrobacterium tumefaciens virulence D2 protein is responsible for precise integration of T-DNA into the plant genome. EMBO J. 14(14): 3585-95.
13. van Attikum H, Bundock P, Overmeer R M, Lee L Y, Gelvin S B, Hooykaas P J. (2003) The Arabidopsis AtLIG4 gene is required for the repair of DNA damage, but not for the integration of Agrobacterium T-DNA. Nucleic Acids Res. 2003 31 (14):4247-55.

What is claimed is:

1. A method for transforming a plant, the method comprising the steps of:
a) providing a transformation-enhancing composition comprising a plant growth nutrient for growing the plant;
b) enriching the plant growth nutrient with an effective amount of at least one nitrogen-containing compound;
c) contacting the plant with the transformation-enhancing composition;
d) transforming a cell from the plant with a nucleic acid of interest; and
e) regenerating a transformed plant from the transformed cell.

2. The method as set forth in claim 1 further comprising the step of enriching the plant growth nutrient with an effective amount of at least one ammonium-containing compound.

3. The method as set forth in claim 1 wherein the nitrogen-containing compound comprises a nitrate compound selected from the group consisting of magnesium nitrate, sodium nitrate, ammonium nitrate, calcium nitrate, barium nitrate, potassium nitrate, hydroxylammonium nitrate, bergilium nitrate, caesium nitrate, chromium nitrate, cobalt nitrate, ferric nitrate, lead nitrate, lithium nitrate, manganese nitrate, mercury nitrate, nickel nitrate, silver nitrate, strontium nitrate, and zinc nitrate.

4. The method as set forth in claim 2 wherein the ammonium-containing compound is selected from the group consisting of ammonium acetate, ammonium bicarbonate, ammonium bromide, ammonium carbonate, ammonium cerium(IV) nitrate, ammonium chlorate, ammonium chloride, ammonium diuranate, ammonium fluoride, ammonium hexachloroplatinate, ammonium hydrogen fluoride, ammonium hydroxide, ammonium iodide, ammonium lauryl sulfate, ammonium nitrate, ammonium nitrite, ammonium perchlorate, ammonium persulfate, ammonium phosphate, ammonium sulfate, ammonium sulfide, ammonium thioglycolate, ammonium uranyl carbonate, ammonium vanadate, diammonium phosphate.

5. The method as set forth in claim 1 further comprises enriching the plant growth nutrient with an effective amount of ammonium nitrate, wherein the concentration of ammonium nitrate in the transformation-enhancing composition is sufficiently greater than 1.65 g/L.

6. The method as set forth in claim 1 wherein the transformation-enhancing composition comprises an effective amount of ammonium nitrate at a concentration greater than 1.65 g/L and less than or equal to 5.00 g/L.

7. The method as set forth in claim 1 wherein the transformation-enhancing composition comprises an effective amount of ammonium nitrate at a concentration greater than 1.65 g/L and less than or equal to 3.30 g/L whereby transformation frequency is enhanced.

8. The method as set forth in claim 1 wherein the transformation-enhancing composition comprises an effective amount of ammonium nitrate at a concentration greater than 1.65 g/L and less than or equal to 3.30 g/L whereby homologous recombination is enhanced.

9. The method as set forth in claim 1 wherein the transformation-enhancing composition comprises an effective amount of ammonium nitrate at a concentration that is greater than 1.65 g/L and less than or equal to 3.30 g/L for reducing deletion of the nucleic acid transformed in the plant.

10. The method as set forth in claim 1 wherein the plant comprises monocots or dicots.

11. The method as set forth in claim 1 wherein the plant comprises Arabidopsis plants.

12. The method as set forth in claim 1 wherein the plant comprises tobacco plants.

13. The method as set forth in claim 1 wherein the plant comprises wheat plants.

14. The method as set forth in claim 1 wherein the plant growth nutrient comprises a plant growth medium.

15. The method as set forth in claim 14 wherein the plant growth medium comprises a Murashige Skoog medium.

16. The method as set forth in claim 1 wherein the transformation step comprises Agrobacterium-mediated transformation.

17. The method as set forth in claim 1 wherein the transformation step comprises bombardment-mediated transformation.

18. A method for enhancing plant transformation frequency, the method comprising the steps of:
a) contacting the plant with an effective amount of a transformation-enhancing composition comprising a plant growth nutrient containing an enriched amount of ammonium nitrate at an effective concentration substantially greater than 1.65 g/L;
b) transforming a cell from the plant with a nucleic acid of interest; and
c) regenerating a transformed plant from the transformed cell.

19. The method as set forth in claim 18 wherein the transformation-enhancing composition comprises ammonium nitrate in an effective amount for enhancing homologous recombination in the plant, the amount being greater than 1.65 g/L and less than 5.00 g/L.

20. The method as set forth in claim 18 wherein the plant comprises monocots or dicots.

21. The method as set forth in claim 18 wherein the plant comprises Arabidopsis plants.

22. The method as set forth in claim 18 wherein the plant comprises tobacco plants or wheat plants.

23. The method as set forth in claim 18 wherein the transformation step comprises Agrobacterium-mediated transformation.

24. The method as set forth in claim 18 wherein the transformation step comprises bombardment-mediated transformation.

25. The method as set forth in claim 18 wherein the plant growth nutrient comprises a plant growth medium.

26. A transformation-enhancing composition for plants comprising:
a) a plant growth nutrient for growing the plants; and
b) an effective amount of at least one nitrogen-containing compound, wherein the combination of the compound with the plant growth nutrient produces a mixture for enhancing plant transformation frequency.

27. The transformation-enhancing composition as set forth in claim 26 further comprises at least one ammonium-containing compound.

28. The transformation-enhancing composition as set forth in claim 27 wherein the nitrogen-containing compound comprises a nitrate compound selected from the group consisting of magnesium nitrate, sodium nitrate, ammonium nitrate, calcium nitrate, barium nitrate, potassium nitrate, hydroxylammonium nitrate, bergllium nitrate, caesium nitrate, chromium nitrate, cobalt nitrate, ferric nitrate, lead nitrate, lithium nitrate, manganese nitrate, mercury nitrate, nickel nitrate, silver nitrate, strontium nitrate, and zinc nitrate.

29. The transformation-enhancing composition as set forth in claim 28 wherein the ammonium-containing compound is selected from the group consisting of ammonium perchlorate, ammonium borate, ammonium carbonate, ammonium chlorate, ammonium chromate, ammonium dichromate, ammonium iodate, ammonium phosphate, ammonium sulfate, ammonium bisulfate, ammonium thiosulfate, ammonium bicarbonate, and ammonium acetate.

30. The transformation-enhancing composition as set forth in claim 29 further comprises an ammonium nitrate in an amount of at least 3.3 g/L.

31. The transformation-enhancing composition as set forth in claim 30 further comprises an ammonium nitrate in an amount ranging from about 1.65 g/L to about 5.00 g/L.

32. The transformation-enhancing composition as set forth in claim 31 wherein the mixture that is produced substantially enhances homologous recombination in the plants.

33. The transformation-enhancing composition as set forth in claim 32 wherein the plants comprise monocots or dicots.

34. The transformation-enhancing composition as set forth in claim 33 wherein the plants comprise Arabidopsis plants.

35. The transformation-enhancing composition as set forth in claim 34 wherein the plants comprise tobacco plants.

36. The transformation-enhancing composition as set forth in claim 35 wherein the plant growth nutrient comprises a standard plant growth medium.

37. The transformation-enhancing composition as set forth in claim 36 wherein the plant growth medium comprises a Murashige Skoog medium.

38. A kit for transforming plants, comprising:
a) a part A containing a plant growth nutrient for growing plants;
b) a part B containing an effective amount of an ammonium nitrate.

39. The kit as set forth in claim 38 further comprises an instruction manual.

40. The kit as set forth in claim 39 wherein the part B comprises ammonium nitrate in an amount greater than 1.65 g/L and less than or equal to 5.00 g/L.

41. Use of ammonium nitrate for enhancing transformation in plants.

42. Use as set forth in claim 41 wherein the plants comprise monocots and dicots.

43. Use as set forth in claim 42 wherein the plants comprise Arabidopsis plants.

44. Use as set forth in claim 43 wherein the plants comprise tobacco plants.

45. Use as set forth in claim 44 wherein at least 3.3 g/L of the ammonium nitrate is used for enhancing homologous recombination in the plants.

46. Use as set forth in claim 45 wherein the ammonium nitrate enhances transformation frequency in the plants.
